(19) United States
(12) Patent Application Publication
(10) Pub. No.: US 2022/0044762 A1
(43) Pub. Date: Feb. 10, 2022

(54) METHODS OF ASSESSING BREAST CANCER USING MACHINE LEARNING SYSTEMS

(71) Applicant: Agendia NV, Amsterdam (NL)

(72) Inventors: Bastiaan Van Der Baan, Irvine, CA (US); Annuska Maria Glas, Irvine, CA (US)

(21) Appl. No.: 17/395,161

(22) Filed: Aug. 5, 2021

Related U.S. Application Data
(60) Provisional application No. 63/062,102, filed on Aug. 6, 2020.

(51) Int. Cl. G16B 25/10; C12Q 1/6886; G16B 40/20; G16H 50/20; G16H 50/70; G16H 50/30; G16H 10/40; G16H 20/10; G16H 15/00; G06N 20/00; G06T 7/00

(52) U.S. Cl. CPC G16B 25/10; G06T 2207/30024; G16B 40/20; G16H 50/20; G16H 50/70; G16H 50/30; G16H 10/40; G16H 20/10; G16H 15/00; G06N 20/00; G06T 7/0012; C12Q 2600/112; C12Q 2600/106; C12Q 2600/158; G06T 2207/30068; G06T 2207/30096; C12Q 1/6886

(57) ABSTRACT

The present disclosure provides methods and systems using machine learning to assess one or more of a patient's biomarkers to analyze various conditions, including cancers, such as breast cancer. The present systems and methods can be trained to analyze patient's biomarker data to form prognoses, diagnoses, and treatment suggestions. Further, the present systems and methods can use biomarker feature data and clinical feature data to create novel correlations in order to provide more accurate, patient-specific diagnoses, prognoses, and treatment suggestions.

### TECHNICAL FIELD

The present disclosure relates to methods and systems for using machine learning and biomarkers to analyze various conditions, including cancers, such as breast cancer.

### BACKGROUND

Breast cancer is the second most common cancer among women in the United States. Despite advances in screening and treatment, breast cancer remains the second leading cause of cancer death among women. Further, recent studies have shown that there are racial/ethnic variations in breast cancer tumor characteristics, subtypes, relative treatment success rates, and recurrence rates. Moreover, the efficacy of various treatments diverges amongst breast cancer subtypes at various stages of progression. This creates a complex picture for pathologists and oncologists in diagnosing, treating, and predicting recurrence in breast cancer patients. Thus, while the aggregate impact of breast cancer is clear, accurate, patient-specific diagnosis, prognosis, and treatment remains relatively obscured.

Although breast cancers come in myriad forms and presentations, they are generally classified based upon histological appearance, i.e., using histopathological practices. This generally means a biopsy followed by a microscopic image analysis. In doing so, a pathologist will often examine whole-slide images (WSI) of tissue scans obtained from the biopsied tissue to type and stage the malignancy. This is a time consuming and difficult process.

Not only are these histopathology assays difficult, but they are often inconsistent due to the “human factor”. Although systematic training and guideline harmonization have been implemented, histopathology relies on the subjective analysis, visual perception, and judgment of individual pathologists. Further compounding this issue is the emergence of minimally invasive methods to obtain samples, which often provide samples of reduced size and/or quality.

Various molecular analysis assays have also been introduced to diagnose, type, and stage breast cancer. Though, in principle, these assays should be more objective than visual histopathology, they come with their own limitations. Breast cancer is a heterogeneous condition, differing between patients and patient populations. Thus, finding definitive biomarkers to characterize the biological drivers of the disease process remains elusive in breast cancer.

Accordingly, there is a need in the art to remove the subjective nature of histopathology assays in characterizing breast cancers. Concurrently, there is a need to better identify biomarkers that can diagnose various breast cancers and biomarkers that provide predictive prognostic measures.

### SUMMARY

The invention provides systems and methods for analyzing diseases, such as breast cancer, using machine learning (ML) in conjunction with biomarker feature data (such as, for example, from whole-slide images of tumor tissue) and clinical feature data (such as, for example, a patient's age and comorbidities). The systems and methods of the disclosure leverage the immense power of ML to analyze vast amounts of data, both old and new, to find new correlations in order to provide predictive outputs that provide accurate and personalized diagnoses, prognoses, and treatment suggestions for diseases, such as breast cancer. Systems of the disclosure are far more powerful and flexible than existing systems that use rule-based program models that require software engineers to code explicit rules, relationships, and correlations. Rather, the systems and methods of the invention can use ML to create novel correlations, such as correlating particular biomarkers with certain subtypes of breast cancer.

Further, over time, as the systems and methods of the invention incorporate data from tested patients, the systems and methods are useful to increase the specificity and sensitivity of analysis from existing clinical tests. For instance, several gene expression panels exist for typing and assessing the risk of patients' breast cancer. As more patients are tested using those panels, data from those tests and/or patient data, such as clinical outcome, are fed into an ML system of described herein. Over time, the system forms new correlations based on the additional volume of data fed into it. Thus, for example, new types of diagnoses and/or prognoses can emerge, including those for particular patient groups, even while using an existing test. Moreover, these new correlations can be used to improve existing tests, for example, by assigning predictive weights to various tested features, e.g., different genes tested by an existing panel.

Additionally, ML has the power to form correlations in an objective, unbiased manner, while using disparate data, such as gene expression and image data. This can improve consistency, for instance, in histopathology which has traditionally relied subjective analysis from pathologists. Further, by combining disparate pieces of information, methods and systems of the inventions create new tests to provide accurate, patient-specific diagnoses, prognoses, and treatment suggestions.

Predictive outputs may include a metric for each type of biomarker feature analyzed. These metrics may be combined or weighted to increase broad diagnostic value. Predictive outputs may include signature biomarker features for specific conditions or classes of conditions, for example, an RNA expression signature for a subtype of breast cancer. Predictive outputs may be used to assess disease severity, such as staging breast cancer or predicting the risk of metastasis, recurrence, or residual disease. The methods and systems of the invention may also provide a report score indicating a risk of cancer recurrence or a distant metastasis event. This can better inform potential treatment options for a particular patient or patient group.

The methods and systems of the disclosure can be used to analyze breast cancer with ML. This can include training an ML analysis system to associate, for example, RNA expression signatures with clinical outcomes. Then, for a particular patient, the expression levels of RNA molecules in a blood sample are determined. These RNA expression levels are used to produce an RNA signature associated with the sample. The signature can be provided to the trained analysis system as an input. The analysis system is then operated to assess the disease, i.e., breast cancer.

Assessing a disease can include one or more of predicting disease severity, determining a diagnosis or stage of disease progression, classifying cancer type, and predicting a drug response. The methods and systems can be used to diagnose a tumor before the tumor is visible. This allows earlier treatment than is provided by existing modalities of diagnosis. The methods and systems of the disclosure may further provide a suggested therapy based on a predicted drug or treatment response. Such treatments may include, for example, a multi-drug therapy or no therapy at all.

The systems and methods of the invention may include or use a computer system hosting a supervised clustering algorithm. Such a clustering algorithm may be used to increase the specificity of diagnosis, such as classifying breast cancer as Luminal, Basal, or HER2.

The systems and methods of the invention can use data from multiple samples or assays to provide biomarker feature data. For example the methods and systems may include providing gene transcription data and genomic data to the analysis system as an input to assess a disease based on a combination of the expression levels and the genomic data. For breast cancer, the genomic data may comprise a mutation status of a BRCA gene. Additional assay data may also be used by the systems and methods of the disclosure to refine a disease assessment. For instance, the methods and systems of the disclosure may include conducting an RNA expression analysis and then analyzing genomic data or an image of tissue from a patient to support or refine a disease assessment. An image may include, for example, an image of a stained FFPE slide from a tumor from the patient.

In methods and systems of the invention, RNA expression levels are determined for RNA molecules in one or more extracellular vesicles isolated from a blood sample. RNA transcripts in extracellular vesicles are protected, and therefore provide a more accurate assessment of expression than free RNA. In methods and systems of the invention, RNA expression levels are determined by measuring transcripts from a pre-determined panel of one or more target genes and one or more reference genes. The RNA expression signature may a weighted metric of levels of the target genes and the reference genes. The target genes or reference genes can be selected for inclusion in the RNA signature when they have average expression levels that are significantly above a pre-determined level of expression associated with background noise.

The disclosure also provides systems and methods that include providing training data to an analysis system. The training data can include, for instance, gene expression signatures and image data with known patient outcomes. The analysis system may be trained to correlate gene expression signatures with image data. Then, RNA expression levels are measured from a patient's blood sample and providing as input to the trained analysis system. The analysis system can be operated to assess disease based on learned correlations between gene expression signatures and the image data. The image data may, for example, include images of stained FFPE slides from tumors. Then, the analysis system can generate a report assessing a risk of a distant metastasis event based on the learned correlations.

### BRIEF DESCRIPTION OF DRAWINGS

FIG. 1 shows a workflow according to the disclosure.

FIG. 2 shows a computer system with a machine learning subsystem.

FIG. 3 illustrates a deep-learning neural network.

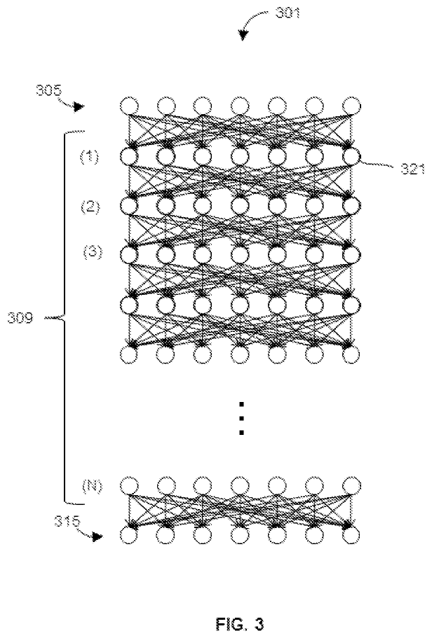

FIG. 4 shows a feature vector in a neural network.

FIG. 5 shows a platform of the disclosure.

### DETAILED DESCRIPTION

The present disclosure relates to methods and systems for analyzing breast cancer using a combination of machine learning (ML) and patient clinical data (e.g., histopathology presentations, biomarkers, and patient data). This includes not only systems and methods using ML, but also the training of ML systems to increase their accuracy and predictive value.

Machine learning is branch of computer science in which machine-based approaches are used to make predictions. (Bera et al., Nat Rev Clin Oncol., 16(11):703-715 (2019)). ML-based approaches involve a system learning from data fed into it, and use this data to make and/or refine predictions. Id. Machine learning is distinct from traditional, rule-based or statistics-based program models. (Rajkomar et al., N Engl J Med, 380:1347-58 (2019)). Rule-based program models require software engineers to code explicit rules, relationships, and correlations. Id. For example, in the medical context, a physician may input a patient's symptoms and current medications into a rule-based program. In response, the program will provide a suggested treatment based upon preconfigured rules.

In contrast, and as a generalization, in ML a model learns from examples fed into it. Id. Over time, the ML model learns from these examples and creates new models and routines based on acquired information. Id. As a result, an ML model may create new correlations, relationships, routines or processes never contemplated by a human. A subset of ML is deep learning (DL). (Bera et al. (2019)). DL uses artificial neural networks. A DL network generally comprises layers of artificial neural networks. Id. These layers may include an input layer, an output layer, and multiple hidden layers. Id. DL has been shown to learn and form relationships that exceed the capabilities of humans. (Rajkomar et al. (2019)).

By combining the ability of ML, including DL, to develop novel routines, correlations, relationships and processes amongst vast data sets of disease biomarker features and patients' clinical data features, the methods and systems of the disclosure can provide accurate diagnoses, prognoses, and treatment suggestions tailored to specific patients and patient groups afflicted with diseases, including breast cancer.

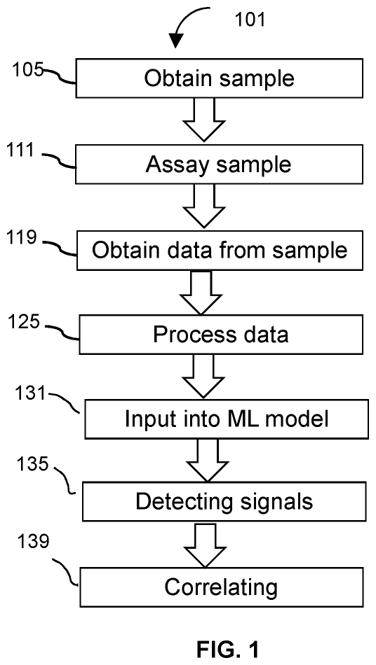

FIG. 1 diagrams a general workflow used in the methods and systems of the present disclosure. The workflow 101 includes obtaining sample 105 from a patient relevant to a particular disease, such as breast cancer. For example, a sample may include a tissue biopsy, a blood draw, and the like. This may include obtaining more than one type of sample from a patient, e.g., a tissue biopsy for a histopathology analysis and a blood draw for an RNA expression analysis. The sample is assayed 111. For example, tissue biopsies may be prepared and stained for a whole-slide image (WSI) and blood draws may be used to isolate and sequence nucleic acids to determine RNA expression levels. Then, relevant data is obtained 119 for any assay completed.

Once data is obtained 119, the data is processed 125. Processing the data 125 transforms the data into signals that can be analyzed by an ML model. For example, if a whole-slide image was obtained, the image is transformed into pixels that can be analyzed by an ML model. Processing the data 125 may also include normalizing or tuning the data. For example, with a WSI, the color and saturation of the image can be adjusted to account for differences arising between imaging instruments or staining procedures. Processing the data 125 may further include annotation. Annotation may, for example, comprise indicating or identifying certain features or areas of interest on a WSI. Annotation may also include clinical feature data relevant to a particular patient, such as age, sex, gender, ethnicity and the like. These annotations may be used by the ML model.

Processing the data 125 may be performed by one or more relevant algorithms and/or by human interaction. Processing 125 may be iterative, such that the data undergoes multiple rounds or methods of processing to fine-tune the data.

After processing the data 125 it is in a format that can be used by an ML model, and includes signals of clinical features that the model analyzes. The processed data is then input into the ML model 131. The ML model analyzes the data to detect relevant signals 135. Detecting signals may include, for example, identifying certain biomarker features, such as the spatial distribution of immune cells on a WSI, which are known to have prognostic value in certain types of breast cancer. The ML model correlates these signals 139 to provide a predictive output. The predictive output may be a predictive diagnosis, prognosis, assignment to a particular risk category, a treatment suggestion, and the like. Based on this predictive output a clinician may undertake an appropriate action, such as a particular course of treatment or non-treatment, monitoring, or subsequent testing.

Predictive outputs may include a metric for each type of biomarker feature analyzed. These metrics may be combined to form a larger prediction. These metrics may be weighted. Predictive outputs may include signature biomarker features for certain types of conditions, for example, an RNA expression signature for a subtype of breast cancer. Predictive outputs may be used to assess disease severity, such as staging breast cancer or predicting the risk of metastasis, recurrence, or residual risk. Predictive outputs may be longitudinal. Longitudinal outputs may be outputs for the same patient or patient population over time, and updated based upon additional biomarker feature data or clinical feature data. Predictive outputs may be based upon threshold values for one or more biomarker features or clinical features. Threshold values may be created by ML models or by humans. ML models may be used to provide predictive outputs for various treatment options for particular patients or patient populations. A single tissue sample may be used to provide biomarker feature data to provide predictive outputs for a patient's risk (e.g., likely risk of metastasis for a tumor), relative treatment efficacies, and benefit of further monitoring (e.g., how often the patient should have a tumor analyzed). A single tissue, e.g., a particular tumor, may be monitored at several time points and analyzed by an ML model to provide continual predictive outputs, including a risk score and treatment score.

RNA expression levels are an important biomarker feature analyzed to diagnose and predict clinical outcomes of diseases and conditions, including breast cancer. The methods and systems of the disclosure use RNA expression levels as a biomarker feature analyzed using machine learning. RNA expression levels have been shown to correlate specific disease types and probable clinical outcomes.

For example, the BluePrint test (Agendia®) is an 80-gene signature assay that measures the combined RNA expression of 80 genes. This test has consistently been able to classify the majority of tested breast cancer patients into definitive breast cancer clinical subtypes, i.e., Luminal-type, Basal-type, and HER2-type. (Mittempergher et al., Translational Oncology, 13 (2020) 100756). For each clinical subtype, a signature RNA expression was determined. A patient's RNA profile is compared to these signature RNA expression levels to determine the patient's clinical subtype. Id. The MammaPrint test (Agendia®) is a 70-gene signature assay that measures the combined RNA expression of 70-genes to assign breast cancer tumors as being of a high or low risk for metastasis. Id. These tests guide a physician's treatment decisions, including whether to pursue early chemotherapy, and avoiding aggressive treatments when they would provide no benefit.

Using machine learning, the systems and methods of the disclosure can expand and improve diagnostic accuracy and prognoses based upon RNA expression levels.

For example, using the BluePrint and MammaPrint test panels, patients' RNA expression profiles and clinical feature data are input into an ML model. Clinical feature data includes, for example, patients' age, sex, ethnicity, comorbidities, treatments, changes in RNA expression over time and in response to treatment, and clinical outcomes, including recurrence. The ML model learns from these data sets and creates novel correlations amongst biomarker feature data and clinical feature data. For example, a correlation between a particular RNA expression profile and the presence/likelihood of a particular form of breast cancer. These novel correlations are used to create predictive outcomes, which may be more accurate for specific patients and patient subgroups. This improves the diagnostic and prognostic value of existing RNA expression tests.

Advantageously, when using established panels, such as BluePrint and MammaPrint, more data is available for ML learning inputs simply due to the vast number of patients tested by the panels. Larger data sets can naturally include, for example, RNA expression profiles taken during different stages of disease progression and profiles taken after patients have undergone varying treatment regimes. Leveraging the correlative power of ML and its ability to digest and learn from vast data sets, diagnostic and prognostic predications become more patient-specific, and thus accurate for individuals.

Additionally, when using established panels, data sets with consistent formats can be created and shared amongst various physicians and researchers. For example, data sets may be provided as electronic case record forms (eCRF). Individual eCRF files may have their information extracted and placed into a Trial Master File (TMF). These data sets may be tailored in such a way to provide ML input data relevant to investigative and clinical trials.

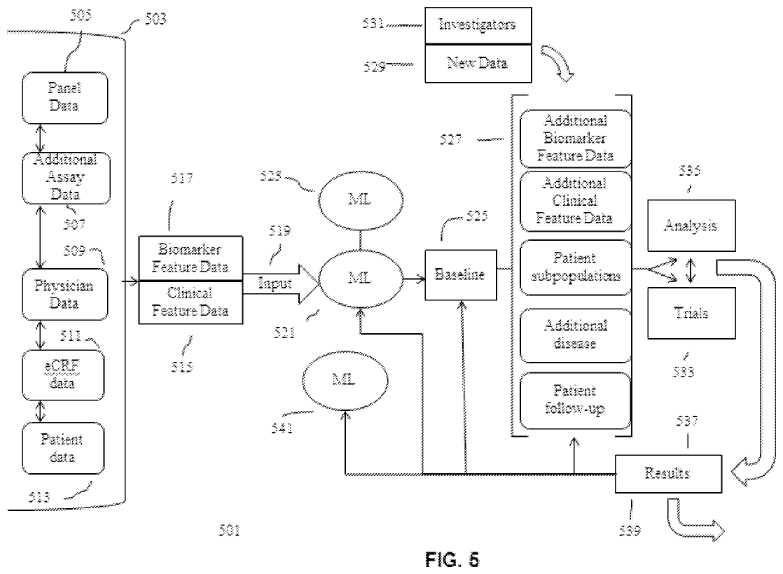

FIG. 5 provides a general overview of a platform of the disclosure through which data, for example, from an established panel, can be leveraged using ML to improve and/or expand an established panel, or be used to create new tests and discoveries.

The platform 501 receives data 503 from several sources, which can include biomarker feature data 517 and clinical feature data 517 from patients. Biomarker feature data from patients may come from an established panel 505 or one or more additional assays 507. This data may be received directly into the platform from, for example, an instrument that processed an established panel. The data may likewise come from physicians 509, such as in the form of an electronic medical record. The data may also come from studies/trials, investigators, researchers and the like, such as in the form of eCRFs 511. Patients may also provide data 513.

Clinical feature data 515, such as patients' age, sex, ethnicity, comorbidities, clinical outcomes, medical treatments and history, patients' familial histories, etc., may be provided by, for example, physicians 509, eCRFs 511, and patients 513, such as through the use of surveys.

Clinical feature data 515 and biomarker feature data 517 are prepared as inputs 519 for an ML model 521. Preparing as inputs 519 may include, for example, processing, normalizing, and annotating the data. This data may be used as data sets to train the ML model 521, or be analyzed by the ML model to provide predictive results. The ML model may train or analyze the data using one or more additional ML models 523. The ML model 521 creates a baseline 525 for providing predictive results. From this baseline 525, patient subsets 527 may emerge or be derived. The patient subsets 527 may include emerge or be derived based upon, for example, relevant new data 529 or actions by one or more investigators 531. Patient subsets 527 may include, for example, subsets according to additional biomarker feature data (e.g., new genomic data), additional clinical feature data (e.g., groups of patients undergoing a specific treatment modality), patient subpopulations (e.g., groups of patients having similar ages, ethnicities, comorbidities, etc.), additional diseases/conditions (e.g., patient groups investigated for the purpose of investigating a disease/condition or who develop a particular disease condition), and patient follow-up (e.g., survey questions, longitudinal monitoring, clinical outcomes, biomarker feature data/clinical feature data over time, etc.). The investigation or emergence of the patient subsets 527 may lead to new trials/studies 533 or analysis 535. Trials/studies 533 and/or analysis 535 may lead to further trials/studies 533 and/or analysis 535. From trials/studies and/or analysis, results 537 can emerge. Results 537 may leave the platform 539, for example, as published studies. Results 537, may also be used to determine further patient subsets 527, update the baseline 525, used as an input to the ML model 521 for analysis and/or training, or put into a different ML model 541 as an input for analysis and/or training.

Further, as RNA expression profiling becomes faster and more ubiquitous, ML can be leveraged to expand existing panels or create new panels. For example, using clinical data inputs and expanded RNA expression profiles, ML can create novel correlations between newly significant genes and clinical data. Similarly, RNA expression profiles from single cells, cellular components, and extracellular components, such as exosomes, can provide more patient-specific predictive correlations. For example, extracellular vesicles have more stable RNA expression profiles relative cells themselves. This is especially important in the heterogeneous and ever-changing environment of a tumor.

RNA expression profiling can also be used with ML to ascertain driver and non-driver (passenger) mutations of a disease or condition, such as breast cancer. Driver mutations provide cancer cells with an “advantage” in growth, proliferation, metastasis, etc. over non-cancer cells. Passenger mutations are those which cancer cells acquired during their evolution into cancer cells, but which do not necessarily provide the cells with an “advantage” over non-cancer cells. Nevertheless, cancer cells will still pass along non-driver mutations during replication. Driver mutations are an obvious choice for forming the basis for diagnosis, prognosis, and treatment targets. However, targeting only cells with a particular driver mutation risks missing cancer cells that lack the particular driver. These cells without the particular driver may cause metastasis or recurrence in their own right. Finding a series of passenger mutations and correlating them using ML with types of cancer and/or driver mutations, can help create more stable and accurate expression signatures of cancers than when considering driver mutations alone.

Moreover, training sets can be used to target particular genes with certain qualities that make them attractive candidates for use in developing robust clinical tests. For example, certain RNA test panels rely on analyzing genes with low expression levels. Analyzing these genes may require several reference genes to be analyzed in parallel to assure normalization. Further, when expression level is low, environmental factors in a clinical setting can disrupt detection. Using ML, the systems and methods of the invention can use training sets targeting genes with high expression levels, or expression levels of a particular threshold. Creating panels with highly expressed genes correlating to a particular disease can improve the accuracy and robustness of a test. Further, by targeting genes with similar expression levels, fewer reference genes are required for normalization. Similarly, training sets can be compiled using genes for which reliable expression testing already exists.

The systems and methods of the disclosure can use genetic screening assays combined with ML to provide predictive diagnoses and prognoses.

Genetics and epigenetics are known to play a key role in a patient's risk for developing a particular disease or condition. For example, certain inherited mutations in the BRCA1 and BRCA2 tumor suppressor genes increase the risk of developing certain types of cancers, including breast cancer. Training an ML system with patients' clinical data and DNA sequence data (biomarker feature data), or portions thereof, an ML system can create novel correlations. For example, ML can be used to correlate one or more alleles, mutations, copy number mutations, etc. with certain features found in patients' clinical feature data. This can be leveraged, for example, to provide predictive outcomes in whether patients will develop a certain condition, how they will respond to various treatments, and predicted clinical outcomes. Genetic and epigenetic data can also be derived from different cancer cells, or from various regions of tumors to ascertain driver and passenger mutations. Further, circulating tumor DNA (ctDNA) can form an important source of biomarker feature data in the methods and systems of the disclosure.

Further, combinations of patients' RNA expression profiles, genetic and/or epigenetic data, and clinical data can be input into an ML system to develop novel correlations leading to predictive outcomes. This can improve the accuracy and functional information of existing tests. Further, like RNA expression panels, specific genetic and epigenetic features with specific qualities may be targeted to create more robust and accurate tests.

Another key biomarker feature to accurately form accurate, diagnoses, prognoses, and treatment decisions, especially in breast cancer, is levels of hormones and hormone receptors. For example, the MammaPrint and BluePrint RNA expression tests, although testing expression levels of many different genes, ultimately classify some types of breast cancer in categories related to hormone receptors, e.g., human epidermal growth factor receptor 2 (HER2) status. The hormonal milieu (e.g., levels of various hormones and hormone receptors) can be used as an input for ML to create correlative signatures to for predictions for diagnoses and prognoses, including risk scores and staging, in breast cancer.

The hormonal milieu can be directly measured for an ML input, e.g., by detecting hormone receptor expression on cells using fluorescence in situ hybridization (FISH). Alternatively, RNA expression of genes related to the hormonal milieu can be used as an ML input. Advantageously, the ability of ML to find correlations between previously unrelated data sets, allows ML to find correlations, for example, between the hormonal milieu and RNA expression of various unrelated genes. Further, ML can combine data from the hormonal milieu to find correlations with, for example, RNA expression and/or genetic and epigenetic data. These new correlations can form the underpinning of diagnoses and prognoses, or simply be used to confirm existing diagnoses or prognoses.

A further key biomarker feature used by the systems and method of the disclosure is imaging data, such as histopathology data, e.g., WSI. WSI has long been used to diagnose breast cancer, including subtypes, stage, and prognoses. However, as WSI has traditionally relied on the subjective examination of pathologists, consistency has been a challenge. Using the objective nature of ML, histopathology for analyzing diseases like breast cancer can be improved using the systems and methods of the disclosure. This includes using ML predictions as a companion to the decision making of trained specialists, or using ML to create independent predictions. Advantageously, ML models can be trained in such a way that they do not have preconceived notions of human specialists, and thus correlate certain image features without the inherent bias of a human.

When for example, analyzing and/or training using WSI, the ML models of the disclosure may use the spatial arrangements and architecture of different types of tissue elements as biomarker feature data. This can include, by way of example, global features of the epithelial and stromal regions, diversity of nuclear shape, orientation, texture, and architecture, glandular architecture, tumor infiltrating lymphocytes, lymphocyte proximity to cancer cells, the ratio of intratumoural lymphocytes to cancer cells, the tumor stroma, etc. During training or analysis, specialists, such as pathologists, may annotate these features prior to input into an ML model. ML may be used to simply more consistently identify these features, or create new correlations between these biomarker features. New correlations may include not only new correlations between biomarker features from an image, but also between biomarker features from other assays, e.g., RNA expression, genetic and epigenetic data, and the hormonal milieu.

ML models of the disclosure can receive tissue images, and known outcomes, to identify features within the images in an unsupervised manner and to create a map of outcome probabilities over the features. The ML models can receive tissue images from patients, identify within the test images predictive features learned from the training steps and locate the predictive features on the map of outcome probabilities to provide a prognosis or diagnosis.

Importantly, image data can be used as a biomarker feature in conjunction with other biomarker features of the disclosure. This finds particular use in longitudinal monitoring of patients/tumors. For example, a tissue sample may be extracted from a patient's tumor, and the extraction imaged (e.g., using WSI). Then, the tissue sample is assayed for biomarker feature data, e.g., RNA expression data. ML can be used to correlate the WSI and the RNA expression data to find correlations between the area of the tumor sampled and the RNA expression data. This process can be iterated over time and/or over different areas of the tumor, to determine, for example, the tumor's response to treatment, to assess the heterogeneous nature of the tumor, to find one or more subtypes of cancer associated with the tumor, and to find the major biologic driver/drivers of the tumor.

Image data can be obtained from tissue samples. Tissue samples may comprise tissue slices harvested from a patient. The tissue slices may contain information regarding the pathological status of the tissue. Alternatively, the image data may comprise images of cells collected by, for example a biopsy, and deposited onto a slide. The cells may include any human cell type, such as, for example, lymphocytes, erythrocytes, macrophages, T-cells, skin cells, fibroblasts, epithelial cells, blood cells, etc.

Detected biomarker features obtained from image data may include a quantification of cell protein expression. Cell protein expression can be quantified from image data based on the detection of a biomarker. ML models of the disclosure can analyze the image data and detect cells positive for certain biomarkers and cells negative for the biomarker based on, for example, pixel intensity and whether the pixel intensity meets a certain threshold. The ML models can then calculate a percentage of cells that are positive for the biomarker. During ML training, these results can be confirmed and compared to those of human specialists viewing the same images.

The systems and methods of the disclosure can include providing an ML model with image data from a tissue sample and operating the machine learning system to detect and annotate features within the image data. The image data can represent a portion, or a subset, of a total image. The ML model can be used to detect and annotate features, such as, for example, cell with certain biomarkers. Annotations can be associated with known pathology states. Annotations made by the machine learning system and annotations made by the reviewers can be correlated so as to validate the capability of the machine learning system to detect those features. The machine learning system can be validated when the correlations meet a certain value.

Processing is a key step, especially with image data as ML inputs. ML can be used in analyzing, for example, whole-cell images. This may include area-based and cell-based analysis of slides or measurements pertaining to tissue aside from cells. Area-based measurements include, for example, quantifying areas with certain stains, vacuoles, and cellular events. Cell-based measurements include, for example, identifying individual cells or subcellular components. However, in order to allow ML to accurately assess these features, processing may be required.

Processing may include “segmenting” an image, which involves spatially parsing an image into constituent portions that may have importance or utility in an analysis. This may include, distinguishing secondary features of a cell from one another, distinguishing benign tissue from that of tumors, distinguishing individual cells from one another, etc. This generally involves transforming an image to pixels, and identifying pixels that represent a particular feature, e.g., a cell. Several algorithms and ML models can be used for segmentation. These can employ adaptive thresholding, watershed segmentation, active contour models, template matching with shape priors, or a probabilistic framework using several strategies.

When using staining, thresholds can be used to identify cells/features. Most simply thresholds can be categorized as 0 (unstained/negative) or +1 (stained). Alternatively, thresholds can be used for a more gradated approach 0 (unstained/negative), +1 (lightly stained), +2 (moderately stained), etc. Additionally, the threshold approach can be used for each color channel (e.g., red, green, blue, yellow) that makes up individual pixels. Individual scores can be combined, with or without channel weighting, to ascertain stained features. This can allow for several types of stain to be used and analyzed on a single image. Further, as instruments and assays may vary between trials and/or institutions, normalization may be used before segmentation to eliminate or reduce bias.

Segmenting may also include parsing images into smaller patches for analysis and/or processing. This may include patches of a particular defined shape.

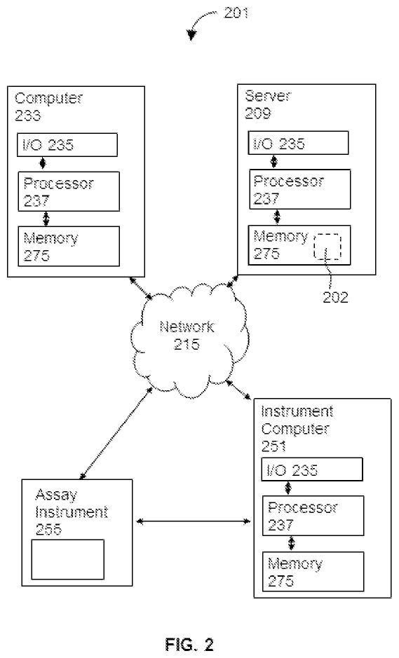

FIG. 2 shows a computer system 201 that may include a machine learning subsystem 202 that has been trained on training data sets. In preferred embodiments, the machine learning subsystem performs the detecting 135. The system 201 includes at least one processor 237 coupled to a memory subsystem 275 including instructions executable by the processor 237 to cause the system 201 to detect 135 relevant signals; and to determine 139 a correlation to provide a predictive output.

The system 201 includes at least one computer 233. Optionally, the system 201 may further include one or more of a server computer 209 one or more assay instruments 255 (e.g., a microarray, nucleotide sequencer, an imager, etc.), which may be coupled to one or more instrument computers 251. Each computer in the system 201 includes a processor 237 coupled to a tangible, non-transitory memory 275 device and at least one input/output device 235. Thus the system 201 includes at least one processor 237 coupled to a memory subsystem 275. The components (e.g., computer, server, instrument computers, and assay instruments) may be in communication over a network 215 that may be wired or wireless and wherein the components may be remotely located or located in close proximity to each other. Using those mechanical components, the system 201 is operable to receive or obtain training data such (e.g., images and molecular assay data) and outcome data as well as test sample data generated by one or more assay instruments or otherwise obtained. The system may use the memory to store the received data as well as the machine learning system data which may be trained and otherwise operated by the processor.

Processor refers to any device or system of devices that performs processing operations. A processor will generally include a chip, such as a single core or multi-core chip (e.g., 12 cores), to provide a central processing unit (CPU). In certain embodiments, a processor may be a graphics processing unit (GPU) such as an NVidia Tesla K80 graphics card from NVIDIA Corporation (Santa Clara, Calif.). A processor may be provided by a chip from Intel or AMD. A processor may be any suitable processor such as the microprocessor sold under the trademark XEON E5-2620 v3 by Intel (Santa Clara, Calif.) or the microprocessor sold under the trademark OPTERON 6200 by AMD (Sunnyvale, Calif.). Computer systems of the invention may include multiple processors including CPUs and or GPUs that may perform different steps of methods of the invention.

The memory subsystem 275 may contain one or any combination of memory devices. A memory device is a mechanical device that stores data or instructions in a machine-readable format. Memory may include one or more sets of instructions (e.g., software) which, when executed by one or more of the processors of the disclosed computers can accomplish some or all of the methods or functions described herein. Preferably, each computer includes a non-transitory memory device such as a solid state drive, flash drive, disk drive, hard drive, subscriber identity module (SIM) card, secure digital card (SD card), micro SD card, or solid-state drive (SSD), optical and magnetic media, others, or a combination thereof.

Using the described components, the system 201 is operable to produce a report and provide the report to a user via an input/output device. An input/output device is a mechanism or system for transferring data into or out of a computer. Exemplary input/output devices include a video display unit (e.g., a liquid crystal display (LCD) or a cathode ray tube (CRT)), a printer, an alphanumeric input device (e.g., a keyboard), a cursor control device (e.g., a mouse), a disk drive unit, a speaker, a touchscreen, an accelerometer, a microphone, a cellular radio frequency antenna, and a network interface device, which can be, for example, a network interface card (NIC), Wi-Fi card, or cellular modem. The machine learning subsystem 202 has preferably trained on training data that includes training images and known marker quantities.

Machine learning systems of the invention may be configured to assay data, and known outcomes, to identify features within assay data in an unsupervised manner and to create a map of outcome probabilities over features in the assay data. The machine learning system can further receive assay data from a test subject, identify within the assay data predictive features learned from the training steps and locate the predictive features on the map of outcome probabilities to provide a prognosis or diagnosis.

Any of several suitable types of machine learning may be used for one or more steps of the disclosed methods. Suitable machine learning types may include neural networks, decision tree learning such as random forests, support vector machines (SVMs), association rule learning, inductive logic programming, regression analysis, clustering, Bayesian networks, reinforcement learning, metric learning, and genetic algorithms. One or more of the machine learning approaches (aka type or model) may be used to complete any or all of the method steps described herein.

For example, one model, such as a neural network, may be used to complete the training steps of autonomously identifying features and associating those features with certain outcomes. Once those features are learned, they may be applied to test samples by the same or different models or classifiers (e.g., a random forest, SVM, regression) for the correlating steps. In certain embodiments, features may be identified and associated with outcomes using one or more machine learning systems and the associations may then be refined using a different machine learning system. Accordingly some of the training steps may be unsupervised using unlabeled data while subsequent training steps (e.g., association refinement) may use supervised training techniques such as regression analysis using the features autonomously identified by the first machine learning system.

In decision tree learning, a model is built that predicts that value of a target variable based on several input variables. Decision trees can generally be divided into two types. In classification trees, target variables take a finite set of values, or classes, whereas in regression trees, the target variable can take continuous values, such as real numbers. Examples of decision tree learning include classification trees, regression trees, boosted trees, bootstrap aggregated trees, random forests, and rotation forests. In decision trees, decisions are made sequentially at a series of nodes, which correspond to input variables. Random forests include multiple decision trees to improve the accuracy of predictions. See Breiman, 2001, Random Forests, Machine Learning 45:5-32, incorporated herein by reference. In random forests, bootstrap aggregating or bagging is used to average predictions by multiple trees that are given different sets of training data. In addition, a random subset of features is selected at each split in the learning process, which reduces spurious correlations that can results from the presence of individual features that are strong predictors for the response variable. Random forests can also be used to determine dissimilarity measurements between unlabeled data by constructing a random forest predictor that distinguishes the observed data from synthetic data. Id.; Shi, T., Horvath, S. (2006), Unsupervised Learning with Random Forest Predictors, Journal of Computational and Graphical Statistics, 15(1):118-138, incorporated herein by reference. Random forests can accordingly by used for unsupervised machine learning methods of the invention.

SVMs are useful for both classification and regression. When used for classification of new data into one of two categories, such as having a disease or not having the disease, a SVM creates a hyperplane in multidimensional space that separates data points into one category or the other. Although the original problem may be expressed in terms that require only finite dimensional space, linear separation of data between categories may not be possible in finite dimensional space. Consequently, multidimensional space is selected to allow construction of hyperplanes that afford clean separation of data points. See Press, W. H. et al., Section 16.5. Support Vector Machines. Numerical Recipes: The Art of Scientific Computing (3rd ed.). New York: Cambridge University (2007), incorporated herein by reference. SVMs can also be used in support vector clustering to perform unsupervised machine learning suitable for some of the methods discussed herein. See Ben-Hur, A., et al., (2001), Support Vector Clustering, Journal of Machine Learning Research, 2:125-137.

Regression analysis is a statistical process for estimating the relationships among variables such as features and outcomes. It includes techniques for modeling and analyzing relationships between a multiple variables. Specifically, regression analysis focuses on changes in a dependent variable in response to changes in single independent variables. Regression analysis can be used to estimate the conditional expectation of the dependent variable given the independent variables. The variation of the dependent variable may be characterized around a regression function and described by a probability distribution. Parameters of the regression model may be estimated using, for example, least squares methods, Bayesian methods, percentage regression, least absolute deviations, nonparametric regression, or distance metric learning.

Association rule learning is a method for discovering interesting relations between variables in large databases. See Agrawal, 1993, Mining association rules between sets of items in large databases, Proc 1993 ACM SIGMOD Int Conf Man Data p. 207, incorporated by reference. Algorithms for performing association rule learning include Apriori, Eclat, FP-growth, and AprioriDP. FIN, PrePost, and PPV, which are described in detail in Agrawal, 1994, Fast algorithms for mining association rules in large databases, in Bocca et al., Eds., Proceedings of the 20th International Conference on Very Large Data Bases (VLDB), Santiago, Chile, September 1994, pages 487-499; Zaki, 2000, Scalable algorithms for association mining, IEEE Trans Knowl Data Eng 12(3):372-390; Han, 2000, Mining Frequent Patterns Without Candidate Generation, Proc 2000 ACM SIGMOD Int Conf Management of Data; Bhalodiya, 2013, An Efficient way to find frequent pattern with dynamic programming approach, NIRMA Univ Intl Conf Eng, 28-30 Nov. 2013; Deng, 2014, Fast mining frequent itemsets using Nodesets, Exp Sys Appl 41(10):4505-4512; Deng, 2012, A New Algorithm for Fast Mining Frequent Itemsets Using N-Lists, Science China Inf Sci 55(9): 2008-2030; and Deng, 2010, A New Fast Vertical Method for Mining Frequent Patterns, Int J Comp Intel Sys 3(6):333-344, the contents of each of which are incorporated by reference. Inductive logic programming relies on logic programming to develop a hypothesis based on positive examples, negative examples, and background knowledge. See Luc De Raedt. A Perspective on Inductive Logic Programming. The Workshop on Current and Future Trends in Logic Programming, Shakertown, to appear in Springer LNCS, 1999; Muggleton, 1993, Inductive logic programming: theory and methods, J Logic Prog 19-20:629-679, incorporated herein by reference.

Bayesian networks are probabilistic graphical models that represent a set of random variables and their conditional dependencies via directed acyclic graphs (DAGs). The DAGs have nodes that represent random variables that may be observable quantities, latent variables, unknown parameters or hypotheses. Edges represent conditional dependencies; nodes that are not connected represent variables that are conditionally independent of each other. Each node is associated with a probability function that takes, as input, a particular set of values for the node's parent variables, and gives (as output) the probability (or probability distribution, if applicable) of the variable represented by the node. See Charniak, 1991, Bayesian Networks without Tears, AI Magazine, p. 50, incorporated by reference.

In preferred embodiments, the machine learning subsystem 202 uses a neural network for the method 101. Preferably, the machine learning subsystem 202 includes a deep-learning neural network that includes an input layer, an output layer, and a plurality of hidden layers.

A neural network, which is modeled on the human brain, allows for processing of information and machine learning. The neural network 301 includes nodes 321 that mimic the function of individual neurons, and the nodes are organized into layers. The neural network includes an input layer, an output layer, and one or more hidden layers that define connections from the input layer to the output layer. The neural network may, for example, have multiple nodes in the output layer and may have any number of hidden layers. The total number of layers in a neural network depends on the number of hidden layers. For example, the neural network may include at least 5 layers, at least 10 layers, at least 15 layers, at least 20 layers, at least 25 layers, at least 30 layers, at least 40 layers, at least 50 layers, or at least 100 layers. The nodes of the neural network serve as points of connectivity between adjacent layers. Nodes in adjacent layers form connections with each other, but nodes within the same layer do not form connections with each other. The neural network 301 has an input layer 305, n hidden layers 309, and an output layer 315. Each layer may comprise a number of nodes 321.

The system may include any neural network that facilitates machine learning. The system may include a known neural network architecture, such as GoogLeNet (Szegedy, et al. Going deeper with convolutions, in CVPR 2015, 2015); AlexNet (Krizhevsky, et al. Imagenet classification with deep convolutional neural networks, in Pereira, et al. Eds., Advances in Neural Information Processing Systems 25, pages 1097-3105, Curran Associates, Inc., 2012); VGG16 (Simonyan & Zisserman, Very deep convolutional networks for large-scale image recognition, CoRR, abs/3409.1556, 2014); or FaceNet (Wang et al., Face Search at Scale: 90 Million Gallery, 2015), each of the aforementioned references are incorporated by reference.

Training data includes data relevant to the assay data which the neural network will analyze, which may be annotated with known outcomes. Nodes in the input layer receive assay data from one or more individuals. For example, the nodes may receive tissue images or portions thereof, such as patches or geometric shapes from within tissue images. The known outcomes associated with the training images are provided to the neural network.

Deep learning (also known as deep structured learning, hierarchical learning or deep machine learning) is a class of machine learning operations that use a cascade of many layers of nonlinear processing units for feature extraction and transformation. Each successive layer uses the output from the previous layer as input. The algorithms may be supervised or unsupervised and applications include pattern analysis (unsupervised) and classification (supervised). Certain embodiments are based on unsupervised learning of multiple levels of features or representations of the data. Higher level features are derived from lower level features to form a hierarchical representation. Those features are preferably represented within nodes as feature vectors.

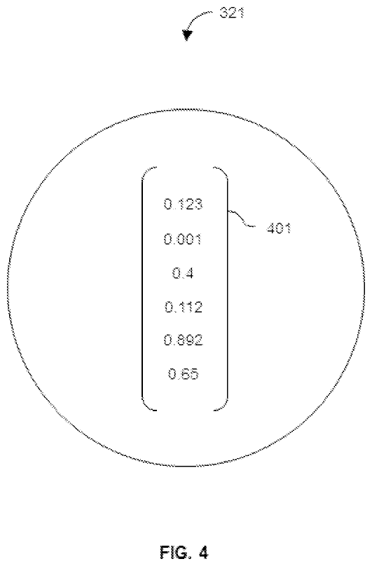

FIG. 4 shows a feature vector 401 representing a feature within a node 321 in a layer of the neural network. Nodes of the neural network may comprise feature vectors 401. Feature vectors may be n-dimensional vectors of numerical features that represent an object. Feature vectors may correspond to pixels, such as in a WSI, and may further represent detected characteristics in the pixels. Feature vectors may be combined with weights using a dot product in order to construct a linear predictor function that is used to determine a score for making a prediction.

Deep learning by the neural network includes learning multiple levels of representations that correspond to different levels of abstraction; the levels form a hierarchy of concepts. In most preferred embodiments, the neural network includes at least 5 and preferably more than 10 hidden layers. The many layers between the input and the output allow the system to operate via multiple processing layers.

Deep learning is part of a broader family of machine learning methods based on learning representations of data. An observation (e.g., an image) can be represented in many ways such as a vector of intensity values per pixel, or in a more abstract way as a set of edges, regions of particular shape, etc. Those features are represented at nodes in the network. Preferably, each feature is structured as a feature vector, a multi-dimensional vector of numerical features that represent some object. The feature provides a numerical representation of objects, since such representations facilitate processing and statistical analysis. Feature vectors are similar to the vectors of explanatory variables used in statistical procedures such as linear regression. Feature vectors are often combined with weights using a dot product in order to construct a linear predictor function that is used to determine a score for making a prediction.

The vector space associated with those vectors may be referred to as the feature space. In order to reduce the dimensionality of the feature space, dimensionality reduction may be employed. Higher-level features can be obtained from already available features and added to the feature vector, in a process referred to as feature construction. Feature construction is the application of a set of constructive operators to a set of existing features resulting in construction of new features.

Within the network 301, nodes 321 are connected in layers, and signals travel from the input layer to the output layer. In certain embodiments, each node 321 in the input layer 305 corresponds to a respective one of the patches from the training data. The nodes 321 of the hidden layer 309 are calculated as a function of a bias term and a weighted sum of the nodes of the input layer, where a respective weight is assigned to each connection between a node of the input layer and a node in the hidden layer. The bias term and the weights between the input layer and the hidden layer are learned autonomously in the training of the neural network. The network 301 may include thousands or millions of nodes 321 and connections. Typically, the signals and state of artificial neurons are real numbers, typically between 0 and 1. Optionally, there may be a threshold function or limiting function on each connection and on the unit itself, such that the signal must surpass the limit before propagating. Back propagation is the use of forward stimulation to modify connection weights, and is sometimes done to train the network using known correct outputs.

The systems and methods of the disclosure may use convolutional neural networks (CNN). A CNN is a feedforward network comprising multiple layers to infer an output from an input. CNNs are used to aggregate local information to provide a global predication. CNNs use multiple convolutional sheets from which the network learns and extracts feature maps using filters between the input and output layers. The layers in a CNN connect at only specific locations with a previous layer. Not all neurons in a CNN connect. CNNs may comprise pooling layers that scale down or reduce the dimensionality of features. CNNs hierarcially deconstruct data into general, low-level cues, which are aggregated to form higher-order relationships to identify features of interest. CNNs predictive utility is in learning repetitive features that occur throughout a data set.

The systems and methods of the disclosure may use fully convolutional networks (FCN). In contrast to CNNs, FCNs can learn representations locally within a data set, and therefore, can detect features that may occur sparsely within a data set.

The systems and methods of the disclosure may use recurrent neural networks (RNN). RNNs have an advantage over CNNs and FCNs in that they can store and learn from inputs over multiple time periods and process the inputs sequentially.

The systems and methods of the disclosure may use generative adversarial networks (GAN), which find particular application in training neural networks. One network is fed training exemplars from which it produces synthetic data. The second network evaluates the agreement between the synthetic data and the original data. This allows GANs to improve the prediction model of the second network.

The outcome data may include information related to a disease or condition. For example and without limitation, the outcome data may include information on one or more of tumor metastasis, tumor growth, or patient survival related to cancer. The cancer may be breast cancer, lung cancer, ovarian cancer, uterine cancer, cervical cancer, and vaginal cancer. The outcome data is from one or more individuals from whom other data, e.g., tissue images have been or will be entered into the machine learning system. In various embodiments the training sets may include image data from patients that are cancer free and the machine learning system may identify features that differentiate between cancer positive and cancer free tissues.

The features detected by the machine learning system may be any quantity, structure, pattern, or other element that can be measured from the training data. Features may be unrecognizable to the human eye. Features may be created autonomously by the machine learning system. Alternatively, features may be created with user input.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

What is claimed is:

1. A method of analyzing breast cancer, the method comprising:
training an analysis system to associate expression signatures with clinical outcomes;
determining expression levels of RNA molecules in a blood sample from a patient;
producing, from the determined expression levels, an RNA signature associated with said sample;
providing the signature as input to the trained analysis system; and
operating the analysis system to assess disease.

2. The method of claim 1, wherein assessing disease comprises one of:
(i) predicting disease severity;
(ii) determining a diagnosis or stage of disease progression;
(iii) classifying cancer type; or
(iv) predicting a drug response.

3. The method of claim 2, wherein the steps of the method are performed to diagnosis a tumor before the tumor is visible.

4. The method of claim 2, further comprising selecting a treatment based on the predicted drug response.

5. The method of claim 4, wherein the treatment comprises a multi-drug therapy.

6. The method of claim 1, wherein the analysis system comprises a computer system hosting a supervised clustering algorithm.

7. The method of claim 6, wherein the clustering algorithm classifies a breast cancer as Luminal, Basal, or HER2.

8. The method of claim 1, further comprising providing genomic data to the analysis system as input and operating the analysis system to assess disease based on a combination of the determined expression levels and the genomic data.

9. The method of claim 8, wherein the genomic data comprises a mutation status of a BRCA gene.

10. The method of claim 8, wherein assessing disease comprises determining a risk of a distant metastatic event and/or selecting a course of treatment.

11. The method of claim 1, further comprising analyzing genomic data or an image of tissue from the patient to support or refine a disease assessment, wherein the image comprises an image of a stained FFPE slide from a tumor from the patient.

12. The method of claim 1, wherein the expression levels are determined for RNA molecules in one or more extracellular vesicles isolated from the blood sample.

13. The method of claim 1, wherein the expression levels are determined by measuring transcripts from a pre-determined panel of one or more target genes and one or more reference genes.

14. The method of claim 13, wherein the RNA signature comprises a weighted metric of levels of the target genes and the reference genes.

15. The method of claim 14, wherein the target genes or reference genes are selected for inclusion in the RNA signature when they have average expression levels that is significantly above a pre-determined level of expression associated with background noise.

16. The method of claim 1, wherein the analysis system reports a score indicating a risk of cancer recurrence or a distant metastasis event.

17. An analysis method comprising:
providing training data to an analysis system, the training data comprising gene expression signatures and image data with known patient outcomes;
training the analysis system to correlate the gene expression signatures with the image data;
measuring RNA expression levels from a patient blood sample and providing the measured expression levels as input to the trained analysis system; and
operating the analysis system to assess disease based on learned correlations between gene expression signatures and the image data.

18. The method of claim 16, wherein the image data comprises images of stained FFPE slides from tumors.

19. The method of claim 16, wherein the analysis system generates a report assessing a risk of a distant metastasis event based on the learned correlations.
